(19) United States
(12) Patent Application Publication
(10) Pub. No.: US 2007/0156456 A1
(43) Pub. Date: Jul. 5, 2007

(54) System for Monitoring Healthcare Related Activity In A Healthcare Enterprise

(75) Inventors: Madelyn McGillin, Wayne, PA (US); Jane E. Marlatt, Linfield, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS MEDICAL SOLUTIONS HEALTH SERVICES CORPORATION, MALVERN, PA (US)

(21) Appl. No.: 11/609,394

(22) Filed: Dec. 12, 2006

Related U.S. Application Data
(60) Provisional application No. 60/756,009, filed on Jan. 4, 2006.

(51) Int. Cl. G06Q 10/00; A61B 5/00

(52) U.S. Cl. 705/2; 600/300

(57) ABSTRACT

A system provides comprehensive patient and resource status information (manpower and equipment required to maintain optimal patient care) in an organization (e.g., a hospital) for use in adjusting resources to meet existing and future conditions. A system for monitoring activity in a healthcare enterprise includes an acquisition interface for acquiring acuity data representative of severity of medical condition of an individual patient, for multiple different patients. A monitoring processor monitors data identifying orders initiated for treatment to be provided to an individual patient and data identifying laboratory test results received for an individual patient, for multiple different patients. A data processor generates data representing status of healthcare activity for multiple patients in response to the data identifying orders and laboratory test results and the acuity data, for multiple different patients. An interface processor provides the data representing status of healthcare activity to a healthcare worker.

This is a non-provisional application of provisional application Ser. No. 60/756,009 by J. E. Marlatt et al. filed Jan. 4, 2006.

### FIELD OF THE INVENTION

This invention concerns a system for monitoring activity in a healthcare enterprise based on acuity data representative of severity of a medical condition, treatment orders and laboratory test results of patients.

### BACKGROUND INFORMATION

In existing healthcare management operations, nurses and other healthcare workers use multiple, non-integrated systems, and multiple manual processes to support clinical supervisory functions. Existing management operations employ electronic staffing systems that maintain staff schedules, competencies and a limited subset of patient acuity (medical condition severity) data. Additionally, electronic bed allocation systems provide a subset of patient data, indicating location, services provided to a patient and fragments of information about patient condition, for example. Existing healthcare management operations do not provide information indicating real time activities of an enterprise, organization or related sub-unit at a glance. Therefore in existing management operations managers typically address staffing issues retrospectively. Issues addressed retrospectively may include, for example, a single nurse station has multiple admissions and discharges per day with ten patient discharges pending with these patients still in their beds awaiting final consultation, e.g., for dietary and wound treatment instructions, while ten new patients are arriving following hospital admissions for the ten open beds, previously noted as being ready. This resource contention is handled by alerting consultation sources to finish the necessary work so that housekeeping can clean rooms before the new patients arrive. In a further example, a patient change in condition, e.g. sudden arrhythmic and rapid heart beat during a MRI procedure, delays a subsequent cardiac catheterization procedure, necessitating staffing changes. In another example, a CAT scan unit is out of service due to equipment failure resulting in multiple critical condition patients being delayed waiting for a CAT unit necessitating retroactive nursing intervention to stabilize the critical patients. Other examples of staffing disruption may include, for example, an Alzheimer patient being missing from an emergency department and wandering the hallways or a portable X-Ray machine cannot be located and bedside examinations are being delayed.

Existing healthcare management operations fail to provide a single system that monitors activities in an entire enterprise to analyze patient condition, transfer status, treatment status and resource (manpower and equipment) status. In existing operations staffing decisions are hampered by incomplete and out of date information and depend upon, for example, nursing manager experience and intuitive decision skills. This results in inconsistent staffing decision results being made across a wide-range of experience and skill levels. The different multiple systems relied on make it difficult to access desired information from remote locations which also delays decisions. A system according to invention principles addresses these deficiencies and related problems.

### SUMMARY OF THE INVENTION

A system provides comprehensive patient and resource status information that visually presents patient level of care, critical risk factors indicating available staffing (numbers and competencies), patient acuity, as well as patient, staff, and equipment location data from clinical and ancillary systems. A system for monitoring activity in a healthcare enterprise includes an acquisition interface for acquiring acuity data representative of severity of medical condition of an individual patient, for multiple different patients. A monitoring processor monitors data identifying orders initiated for treatment to be provided to an individual patient and data identifying laboratory test results received for an individual patient, for multiple different patients. A data processor generates data representing status of healthcare activity for multiple patients in response to the data identifying orders and laboratory test results and the acuity data, for multiple different patients. An interface processor provides the data representing status of healthcare activity to a healthcare worker.

### BRIEF DESCRIPTION OF THE DRAWING

FIG. 1 shows a system for monitoring activity in a healthcare enterprise, according to invention principles.

FIG. 2 shows a diagram indicating functional operation of the system for monitoring activity in a healthcare enterprise, according to invention principles.

FIG. 3 shows a flowchart of a process used in monitoring activity in a healthcare enterprise, according to invention principles.

FIGS. 4 and 5 show user interface display images illustrating patient and resource status information, according to invention principles.

FIG. 6 shows a flowchart of a process used in navigating image menus employed in the activity monitoring system, according to invention principles.

### DETAILED DESCRIPTION OF INVENTION

A system for monitoring activity in a healthcare enterprise provides comprehensive patient and resource status information in a graphical presentation indicating patient level of care, critical (and other) risk factors and resource status (manpower and equipment required to maintain optimal patient care) in an organization (e.g., a hospital). This enables a clinical operation manager to identify and focus on critical decisions. A remote monitoring function enables an organization to view graphically presented patient and resource status information from any location. The system acquires and collates available staffing information (numbers, competencies), patient acuity information and location (e.g. RFID derived) data identifying location of patients, staff, and equipment from clinical and ancillary systems using both push and pull methods. The system employs rules and a workflow processor, to monitor patient, nurse, and equipment events so that adverse actions are minimized.

User interface display images providing graphically presented patient and resource status information also include user selectable interactive icons enabling user access to additional relevant data by expanding or contracting a display image window, for example. The system employs a repository including searchable audit history information enabling a user to access a history of alerts generated for user specified time periods (shift, day, week, month), location, or type of modality (e.g., MRI, CT scan, Ultrasound, radiotherapy, X-ray) equipment. The audit history information supports documentation and risk mitigation and (Joint Commission on Accreditation of Healthcare Organizations) standards reviews. The patient and resource status information supports continuously monitoring critical nursing factors to present contemporaneous nursing requirements for prompting decision makers to adjust resources to meet current resource requirements.

The system supports remote monitoring of off-site locations to provide nurse managers with comprehensive contemporaneous online, real time resource information providing an early warning of an emerging critical event so that a nursing clinical manager can intervene using accurate and current clinical information. The system provides a nurse manager with alerts on mobile devices, such as PDAs, cell phones, pagers, laptop computers, and similar wired or wireless processing devices and enables a user of these processing devices to manage resources to improve clinical operations. A rules and workflow processing function enables a user to determine conditions for generating alerts specific to user needs. For example, in response to satisfaction of the condition (Nurse station <> “SURG10” and patient condition=“poor” and WBC (white blood count) laboratory test result=“critical” and temperature range is between 101 and 106 F), the system changes a patient icon to red. This red alert provides a nurse manager information to prompt initiating a post surgical infection protocol for the patient. The system provides a variable data navigational zoom capability facilitating zoom-in focus on critical events of an individual patient or zoom-out focus at a macro-level for viewing resource status of an organization. Different selectable user interface operation modes present data from multiple different data sources including, for example, staffing information for a care unit or types of patients with similar diagnoses within a unit.

The system enables analysis of stored audit data indicating alert events to provide an authorized manager information about events for user specified periods of time and types of care units. Thereby a manager is able to view activity occurring while a hospital, department or care unit is in a crisis, for example, a fire or other type of emergency, once the hospital, department or care unit returns to normal state. The audit data analysis determines whether healthcare worker actions resulted in maintaining the condition of patients, improving the condition or degrading the condition. The system supports proactive decision-making where clinical managers are seeing the same data at the same time, irrespective their location in a facility and facilitates diagnosing systemic problems using detailed audit information concerning events. The system further provides detailed information associated with critical events, such as alerts, ventilator alarms, and alarms of other monitoring devices, per shift and or per day, for example.

An executable application as used herein comprises code or machine readable instruction for implementing predetermined functions including those of an operating system, healthcare information system or other information processing system, for example, in response user command or input. An executable procedure is a segment of code (machine readable instruction), sub-routine, or other distinct section of code or portion of an executable application for performing one or more particular processes and may include performing operations on received input parameters (or in response to received input parameters) and provide resulting output parameters. A processor as used herein is a device and/or set of machine-readable instructions for performing tasks. As used herein, a processor comprises any one or combination of, hardware, firmware, and/or software. A processor acts upon information by manipulating, analyzing, modifying, converting or transmitting information for use by an executable procedure or an information device, and/or by routing the information to an output device. A processor may use or comprise the capabilities of a controller or microprocessor, for example. A display processor or generator is a known element comprising electronic circuitry or software or a combination of both for generating display images or portions thereof. A user interface comprises one or more display images enabling user interaction with a processor or other device. Workflow comprises a sequence of tasks performed by a device or worker or both.

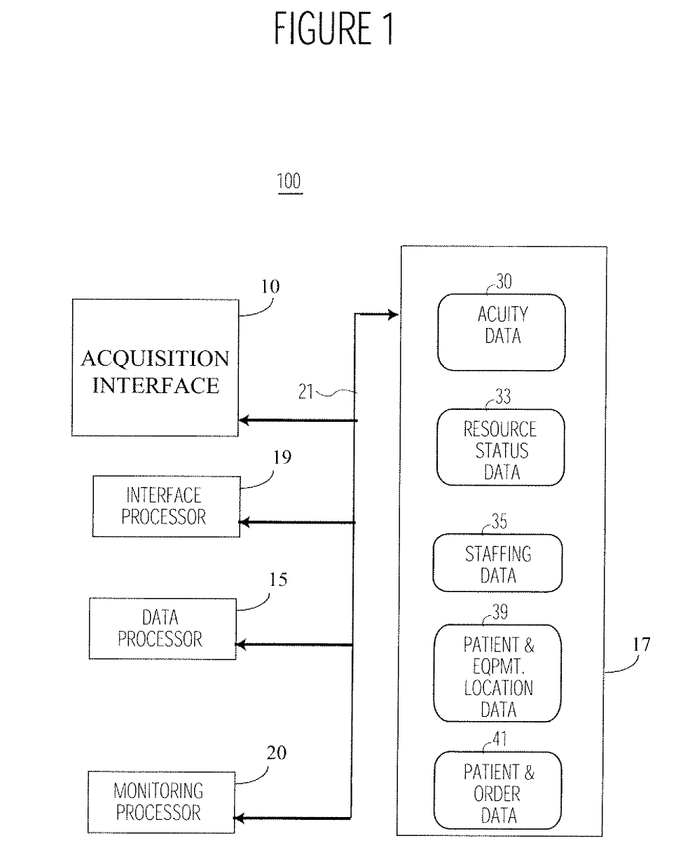

FIG. 1 shows system 100 for monitoring activity in a healthcare enterprise including units 10, 15, 17, 19 and 20 that inter-communicate via communication path 21. Acquisition interface 10 acquires acuity data 30 representative of severity of medical condition of an individual patient, for multiple different patients from different sources including repository 17. Monitoring processor 20 monitors data identifying orders 41 initiated for treatment to be provided to an individual patient and data identifying laboratory test results 41 received for an individual patient, for multiple different patients. The monitoring processor 20 identifies orders initiated for treatment to be provided to an individual patient and data identifying laboratory and radiology test results. Data processor 15 generates data representing status of healthcare activity for multiple patients in response to the data identifying orders and laboratory and radiology test results 41 and acuity data 30, for multiple different patients. Interface processor 19 provides data 33 representing status of healthcare activity to a healthcare worker. Data processor 15 in another embodiment generates the data 33 representing status of healthcare activity using resource status data, staffing data 35 (including availability and competencies) and patient and equipment location data 39.

Interface processor 19 provides the data representing status of healthcare activity to the healthcare worker via a composite single display image (or in another embodiment multiple different images) identifying status of treatment related activity of at least one of, (a) groups of patients, (b) hospital departments, (c) care units and (d) nursing locations, using a visual attribute indicating relative need for additional resources. The display image identifies status of treatment related activity and indicates degrees of available treatment related activity capacity in different healthcare enterprise locations and for different categories (e.g., capacity, acuity, treatment delay) of operational characteristics. The different categories of operational characteristics may also comprise, delay in administering medication or intra-venous infusions, patient discharge delays, nurse call activity or alerts generated. The visual attribute comprises, color, highlighting, a symbol, shape, text or shading.

The system 100 may be employed by any type of enterprise, organization, or department, such as, for example, providers of healthcare products and/or services responsible for servicing the health and/or welfare of people in its care. For example, the system 100 represents a healthcare information system. A healthcare provider provides services directed to the mental, emotional, or physical well being of a patient. Examples of healthcare providers include a hospital, a nursing home, an assisted living care arrangement, a home health care arrangement, a hospice arrangement, a critical care arrangement, a health care clinic, a physical therapy clinic, a chiropractic clinic, a medical supplier, a pharmacy, a doctor's office, and a dental office. When servicing a person in its care, a healthcare provider diagnoses a condition or disease and recommends a course of treatment to cure the condition, if such treatment exists, or provides preventative healthcare services. Examples of the people being serviced by a healthcare provider include a patient, a resident, a client, and an individual.

The system 100 may be fixed and/or mobile (i.e., portable). The system 100 may be implemented in a variety of forms including, but not limited to, one or more of the following: a personal computer (PC), a desktop computer, a server, a laptop computer, a workstation, a minicomputer, a mainframe, a supercomputer, a network-based device, a personal digital assistant (PDA), a smart card, a cellular telephone, a pager, and a wristwatch. The system 100 and/or elements contained therein also may be implemented in a centralized or decentralized configuration. The system 100 may be implemented as a client-server, web-based, or stand-alone configuration.

The communication path 21 (otherwise called network, bus, link, connection, channel, etc.) may use any type of protocol or data format. The protocol or data format includes, but is not limited to, one or more of the following an Internet Protocol (IP), a Transmission Control Protocol Internet protocol (TCPIP), a Hyper Text Transmission Protocol (HTTP), an RS232 protocol, an Ethernet protocol, a Medical Interface Bus (MIB) compatible protocol, a Local Area Network (LAN) protocol, a Wide Area Network (WAN) protocol, a Campus Area Network (CAN) protocol, a Metropolitan Area Network (MAN) protocol, a Home Area Network (HAN) protocol, an Institute Of Electrical And Electronic Engineers (IEEE) bus compatible protocol, a Digital and Imaging Communications (DICOM) protocol, and a Health Level Seven (HL7) protocol.

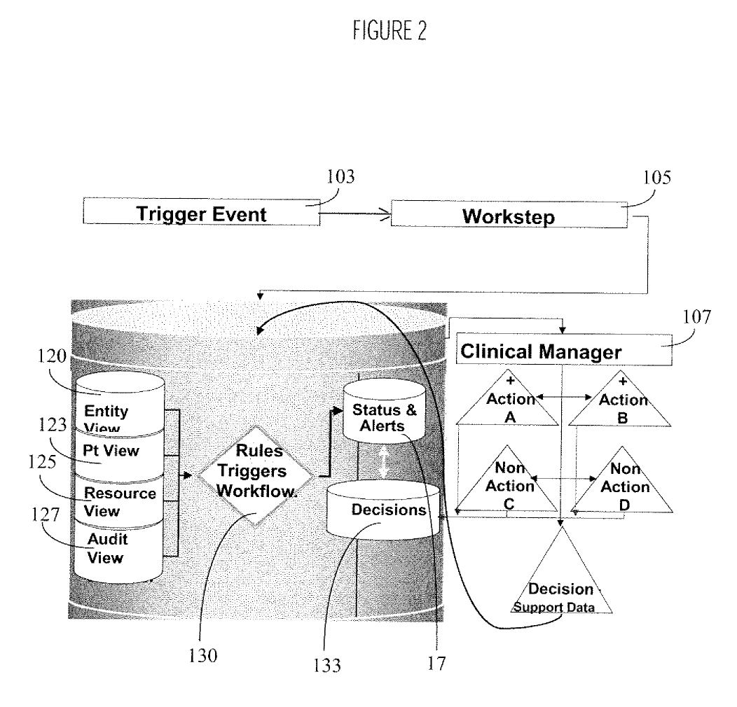

FIG. 2 shows a diagram indicating functional operation of the system for monitoring activity in a healthcare enterprise. A workstep process function 105 generates a display image identifying status of treatment related activity including patient and resource status for an enterprise (e.g., a hospital) and alert notification messages to workers, in response to a clinical trigger event 103. Clinical trigger event 103 may comprise an order for a patient treatment, a documentation data element entry (or observation), entry of patient test result data (generally via an interface from a device or ancillary system), or a workflow engine subprocess. A configuration processor in data processor 15 (FIG. 1) enables a user to enter data identifying types of permitted clinical trigger event 103. The entered clinical trigger event identification data identifies a clinical problem (e.g., on a problem list), a diagnosis on a diagnosis list, or another data element for use in workstep management. Workstep function 105 generates data representing expected (or recommended) actions or decisions based on the patient treatment related activity status and resource (equipment and staffing) status. The actions or decisions initiate predetermined work effort coordination steps (e.g., clinical protocol or a guideline requiring a decision to be made by a clinician). Workstep function 105 also generates ancillary data that provides information regarding a type of clinician and privileges and experience required to accomplish expected (or recommended) actions or make expected decisions.

Workstep function 105 determines in real-time, available clinicians with the required privileges and competencies. Workstep function 105 coordinates patient treatment urgency and priority, with clinician privileges and competencies in providing data indicating available clinicians. Workstep 105 is configurable by a user to automatically assign the most available clinician to perform a particular task or to communicate data indicating available clinicians to a worker (such as a nursing manager or a medical director) to manually choose an appropriate clinician. Workstep 105 automatically assigns the most available clinician to perform a particular task and notifies the clinician by a method selected in accordance with the priority of a particular task. A configuration function in data processor 15 enables a user to enter data associating individual healthcare workers with notification methods (such as mail, pager, email, phone) ranked according to task priority. A notified clinician is provided by unit 107 with actionable information and takes one of the suggested actions (e.g., action A or B) or determines not to act and provides a reason (e.g., non-action C or D). A clinician is provided actionable information (and initiates actions) via multiple user navigable display images presented via user interface 19 (FIG. 1). Workflow function 130 uses actionable information from unit 107 together with treatment decision, order and generated alert information as well as staffing, resource, acuity, and location data identifying location of patients and equipment in repositories 17 and 133, to generate display images including entity view 120, patient view 123, resource view 125 and audit view 127, in response to a clinical trigger event 103.

Entity image view 120 presents information identifying status of treatment related activity including patient and resource status for an enterprise (e.g., a hospital) and alert notification messages to workers. Patient image view 123 presents information identifying status of treatment related activity status and associated resource status for one or more selected patients and alert notification messages to workers associated with the selected patients. Resource image view 125 presents information identifying status of resources including workers and equipment. Audit image view 127 provides stored audit data indicating alert events for a user specified period of time and/or type of care unit. Workflow processor 130 also monitors performance of tasks by assigned clinicians and stores monitoring data (including the number and type of procedures performed and corresponding outcomes and associated data) in repository 133. The system is usable in real-time processes for re-direction of tasks and is of particular use within clinical care settings to coordinate the effectiveness and efficiency of human resources to improve throughput.

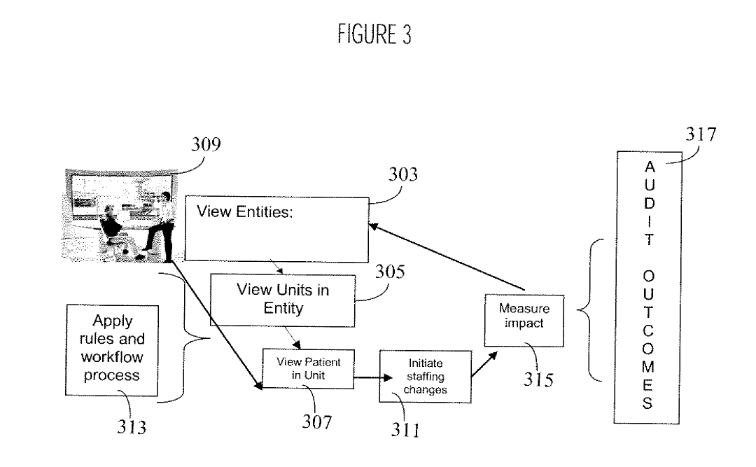

FIG. 3 shows a flowchart of a process performed by system 100 (FIG. 1) used in monitoring activity in a healthcare enterprise to provide treatment related activity, alerts and resource status for an enterprise (e.g., a hospital). System 100 provides a hospital patient and resource management system that monitors clinical and ancillary data and visually represents this data. The data monitored includes patient level of care (acuity), delay in treatment, critical risk factors and resource status (manpower and equipment). User interface 19 provides multiple user selectable and configurable image views that are navigable in a user friendly fashion to provide a user with desired information. User interface 19 provides patient and resource status information for a hospital, for example, in entity image view 303. A user is able to navigate successively from image view 303 to image view 305 showing status information of a unit within an entity and to image view 307 concerning a particular patient within a care unit and providing live video information 309 of a patient, for example. Image views 303, 305 and 307 employ status information derived by applying workflow processes and predetermined rules 313.

A user is able to initiate staffing changes 311 such as activating part time staff to cope with an increase in patient load via image view 307, for example. An auditing process 317 analyzes stored audit data to measure treatment impact 315 to determine whether healthcare worker actions resulted in maintaining the condition of patients, improving the condition or degrading the condition. The audit data including treatment outcomes and alert events is analyzed for user specified periods of time and different types of care units. Treatment outcome analysis results are used to improve status information processing and change image views 303, 305 and 307.

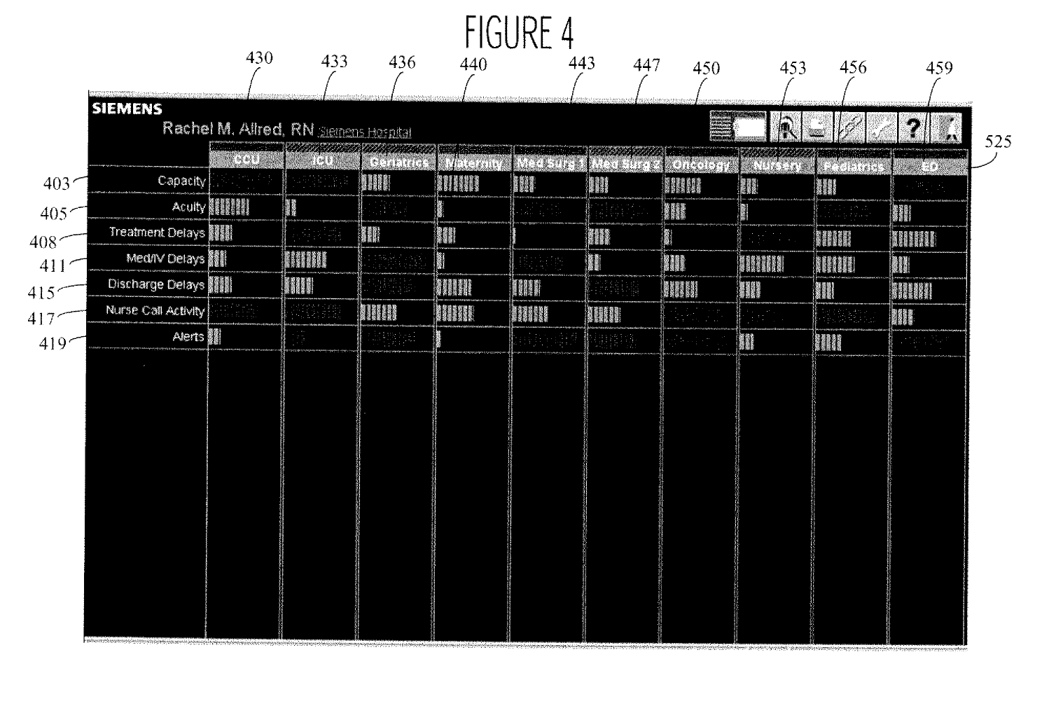

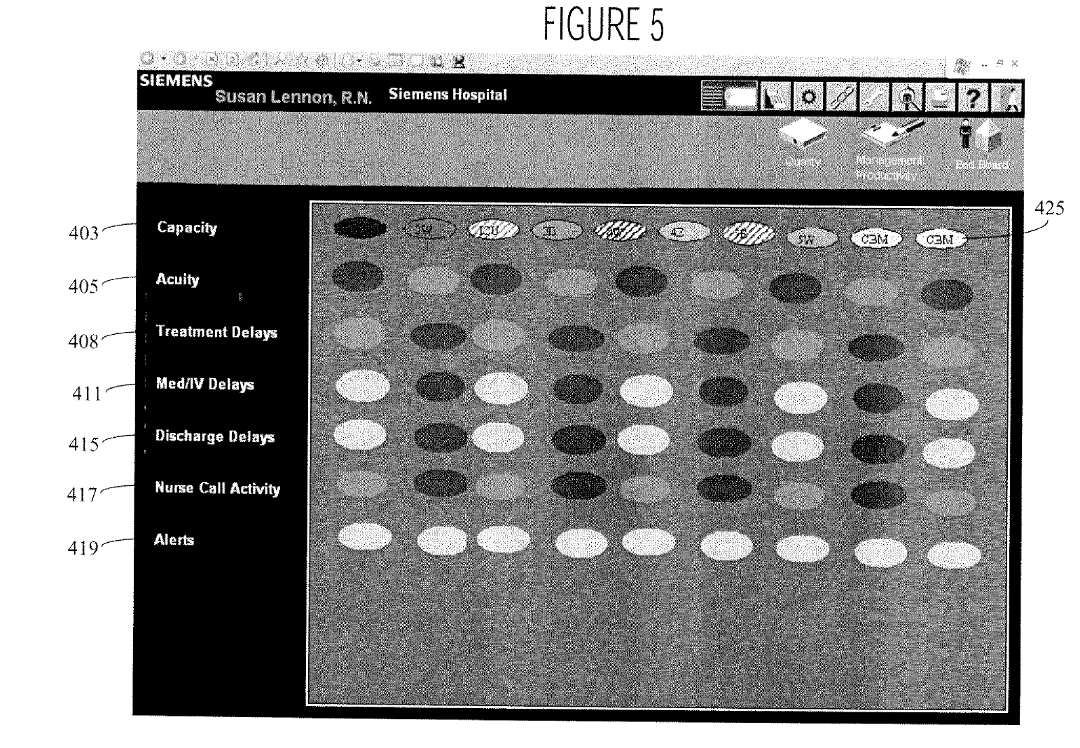

FIGS. 4 and 5 show user interface display images presented by user interface 19 (FIG. 1) illustrating patient and resource status information. The display images are color-coded and in one embodiment use visual attributes such as patterns including stripes, to indicate degrees of capacity. System 100 monitors nurse station capacity, patient aggregate acuity, transfer activity and delays, alarms, and medical alerts, such as critical test values. System 100 uses a Clinical Information System data processor 15 incorporating a Workflow Engine and a Rules Engine to track activity for a nurse manager. A user navigates to access more detailed information by clicking a specific image area or icon. One display image presents critical factors at a nurse station. System 100 uses push and pull data distribution functions to communicate warnings to a specific nurse manager, for example, so that whenever a critical factor turns yellow or red, an alert is wirelessly communicated via a network to a portable device. The nurse manager is able to evaluate the situation, and if necessary initiate orders or commands using system 100 to reassign resources are to close critical resource gaps, as one example.

Monitoring processor 20 continually monitors various hospital processes, including hospital (or department) patient handling capacity, Acuity, Treatments and delays, Med/IV delays, Discharge Delays, Nurse Call Activity and Alerts to evaluate if a process is performing within acceptable under-load or overload thresholds. System 100 employs statistical estimation and inference modeling to establish patient handling and resource under-load and overload thresholds and determine a probability a hospital process is likely to exceed a threshold in a particular period. System 100 determines multiple factors and indicators that are combined into an overall score. The score is compared with an overall threshold defined for a hospital or hospital unit or healthcare unit. Icons in user interface image windows that illustrate patient and resource status information in FIGS. 4 and 5 employ a visual attribute to indicate status characteristics. A color attribute, for example, may be used to visually represent status of a process being monitored whereby, green indicates healthy status, yellow indicates deteriorating status and red indicates critical status.

System 100 uses data from clinical and ancillary systems including staffing data (available staff numbers, staff competencies, credentials, privileges), acuity management data, treatment and treatment delays, discharge delays, and other data. System 100 supports continuous or intermittent monitoring comprising remote monitoring allowing off-site centralized administrative staff to identify and act upon a problem or to prevent a problem from occurring. System 100 provides online and real time data enabling a user to identify a critical area and intervene before a problem occurs. A user is alerted to a patient handling or resource status problem via a display image generated by user interface 19 and is able to initiate an action via the display image to address the problem.

User interface display image FIG. 4 presented on interface 19 (FIG. 1) illustrate patient and resource status information provided by system 100 for hospital locations identified in row 525 including CCU (critical care unit) 430, ICU (intensive care unit) 433, Geriatrics 436, Maternity 440, Med. Surgery 1 443, Med. Surgery 2 447, Oncology 450, Nursery 453, Pediatrics 456 and ED (emergency department) 459. Specifically, FIG. 4 illustrates patient and resource status information for various hospital processes using color-coded graphical icons comprising patterns such as stripes, to indicate degrees of capacity with green indicating healthy status, yellow indicating deteriorating status and red indicating critical status. The color-coded graphical icons indicate status of hospital processes (operational characteristics) including patient handling capacity 403, patient Acuity 405, Treatments and delays 408, Med/IV delays 411, patient Discharge Delays 415, Nurse Call Activity 417 and Alerts 419 for the hospital locations identified in row 525. A capacity icon represents a healthcare unit (e.g., nursing station, ward etc.) overall capacity based on remaining factors of acuity, treatment delays, med/iv delays, patient discharge delays, nurse call activity and alerts. FIG. 5 similarly illustrates patient and resource status information for the hospital processes for locations identified by overlay in row 425 using color-coded graphical icons to indicate capacity with green indicating healthy status, yellow indicating deteriorating status and red indicating critical status.

A user is able to select a capacity icon via a FIG. 4 or 5 image for a desired nursing unit, for example and launch a detailed image view of status information of the selected unit. A Clinical manager uses the FIG. 4 image to view capacity for the ten units she is responsible for managing, for example. The clinical manager determines via FIG. 4 that CCU, ICU and ED (red capacity icons) have exceeded capacity whereas the Geriatrics, Med. Surgery 1, Med. Surgery 2, Nursery and Pediatrics (green capacity icons) are within a desired capacity for the monitored hospital processes. Maternity and Oncology (yellow icons) indicate a deteriorating capacity situation. A clinical manager is provided with detailed status information concerning a selected unit in response to selection of a specific unit capacity icon. An acuity icon represents the acuity (severity of medical condition) of the patients in each unit. Acuity is determined using patient risk factors, current physiological and functional status and ordered services. The FIG. 4 display image enables a clinical manager to quickly see overall acuity of a hospital unit and identify patient care units needing additional resources and to examine detailed acuity data of patients in a specific unit in response to user selection of a care unit acuity icon. In a similar manner to the capacity and acuity Graphical icons, the graphical icons used for Treatment delays, Med/IV delays, delays in patient Discharge, Nurse Call Activity and Alerts indicate status of these hospital processes. Further, user selection of an Alert Icon for a specific unit enables a user to view alerts for physiological monitors, ventilators, ECG monitors, pumps and other electronic devices in the care unit.

In an example of operation, a manager is one of three nursing supervisors on an 11 am-7 pm work shift, managing a busy 1,200 bed tertiary care hospital with 30 patient care areas (wards). The manager is responsible for a group of 10 patient care areas. It is the responsibility of the manager to dispatch relief reserve (re-assignable) staff and management staff to support situations where operational capacity is met or exceeded. The manager receives a message that patient Acuity in the unit Med. Surgery 1 has exceeded the threshold for the current staffing. In response, the manager accesses the system 100 using a PC, notebook or PDA. The manager uses system 100 to determine status of parameters for the unit Med Surgery 1 including delays in treatments, med/IV, discharge, patient call activity and alerts. The manager uses the data from Med Surgery 1 and the other units to make decisions regarding re-assignment of staff. In contrast, in existing known operations the re-assignment of the reserve staff typically occurs too late, arriving after capacity saturation and the reserve staff is unable to provide real assistance. Of equal concern, is the fact that often overloaded nursing staff are too busy providing care to assess and identify a change in the patient acuity and notify management that additional support is needed.

Application Service Provider (ASP) centers and call centers may use the system and processes of FIGS. 1-6 to improve the monitoring of critical components. The real time on-line patient specific status information supports an organization in making planning decisions and the aggregated status data displayed by user interface 19 provides a manager with access to pertinent status data in one location. System 100 employs configurable rules and workflow to enable an organization to set trigger alerts based on customized specific criteria and displays pertinent data at the right time. This is achieved by processor 20 monitoring critical factors involved in nurse staffing using workflow and rules to improve the quality and responsiveness of nursing provided to patients in acute care hospitals. In an example of operation, a nurse supervisor is assigned to five patient care units with responsibility for 250 patients. The patients on these units are critically ill with diagnoses ranging from heart failure, myocardial infarction to traumatic brain and spinal cord injury. A majority of patients are on continuous ventilation and monitoring devices. Bedside radiological and ultrasound diagnostic services are typically utilized given the acuity of the patients. Acuity data is collected via assessment data which classifies and quantifies the level of care required by individual patients and time, order and result data is concurrently monitored to identify acuity status exceeding a predetermined care unit threshold in response to a received item of data or combination of data. System 100 notifies a nurse supervisor (e.g., via a mobile device) when the threshold is met or approached. The nurse supervisor uses the system to look at status data of the nursing units to identify areas able to release staff to assist in managing the increase in patient acuity in other areas. In a multi-facility hospital a remote site uses system 100 for continuous monitoring of the patient and resource data. The continuous monitoring enables management staff to identify problems and whether a patient is likely to need assistance or a process is about to fail and provide timely warning and intervention.

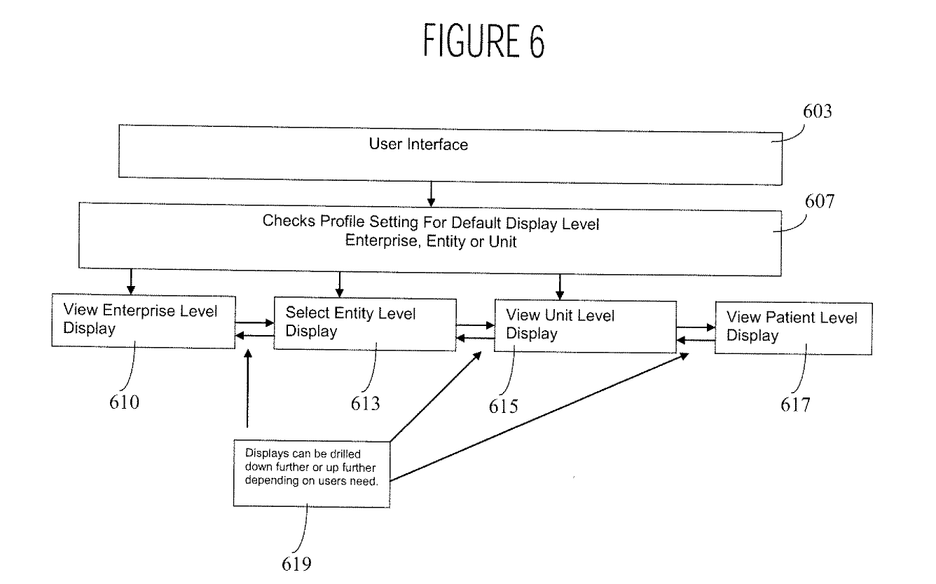

FIG. 6 shows a flowchart of a process used in navigating image menus employed in the activity monitoring system. In response to a command received from user interface 603 to access a patient and resource status display, system 100 in step 607 determines from a system configuration profile if a default display is set to display enterprise (e.g., hospital), entity (e.g., facility such as radiology, laboratory, surgery or a department) or unit (e.g., nurse station, ward or patient room) status data. In response to this determination, user interface 19 presents enterprise 610, entity 613, unit 615 or individual patient 617 status data. A user is able to navigate 619 upwards or downwards between the status display images by command via a browser or other interface, fore example.

The system and processes presented in FIGS. 1-6 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. A system according to invention principles provides personnel and resource status information that visually presents personnel status, critical risk factors indicating available staffing (numbers and competencies), as well as patient, staff and equipment location data from ancillary systems. Further, any of the functions and steps provided in the system of FIG. 1 or processes in FIGS. 2-6 may be automatically implemented in hardware, software or a combination of both and may reside on one or more processing devices located at any location of a network linking the FIG. 1 elements or another linked network including another intra-net or the Internet.

What is claimed is:

1. A system for monitoring activity in a healthcare enterprise, comprising:
an acquisition interface for acquiring acuity data representative of severity of medical condition of an individual patient, for a plurality of different patients;
a monitoring processor for monitoring data identifying orders initiated for treatment to be provided to an individual patient and data identifying laboratory test results received for an individual patient, for a plurality of different patients;
a data processor for generating data representing status of healthcare activity for a plurality of patients in response to said data identifying orders and laboratory test results and said acuity data, for a plurality of different patients; and
an interface processor for providing said data representing status of healthcare activity to a healthcare worker.

2. A system according to claim 1, wherein
said interface processor provides said data representing status of healthcare activity to said healthcare worker via a display image identifying status of treatment related activity of at least one of, (a) groups of patients, (b) hospital departments, (c) care units and (d) nursing locations, using a visual attribute indicating relative need for additional resources.

3. A system according to claim 2, wherein
said visual attribute is at least one of, (a) color, (b) highlighting, (c) a symbol, (d) shape, (e) text and (f) shading.

4. A system according to claim 2, wherein
said visual attribute indicates status of healthcare activity by indicating degrees of available capacity.

5. A system according to claim 1, wherein
said interface processor provides said data representing status of healthcare activity to said healthcare worker via a display image identifying status of treatment related activity in different healthcare enterprise locations and for different categories of operational characteristics.

6. A system according to claim 5, wherein
said different categories of operational characteristics comprise at least two of, (a) capacity, (b) acuity and (c) treatment delay.

7. A system according to claim 5, wherein
said different categories of operational characteristics comprise at least one of, (a) delay in administering medication or intra-venous infusions, (b) patient discharge delays, (c) nurse call activity and (d) alerts generated.

8. A system according to claim 1, wherein
said monitoring data identifies orders initiated for treatment to be provided to an individual patient and data identifying laboratory and radiology test results and
said data processor generates data representing status of healthcare activity for a plurality of patients in response to said data identifying orders and laboratory and radiology test results.

9. A system for monitoring activity in a healthcare enterprise, comprising:
an acquisition interface for acquiring acuity data representative of severity of medical condition of an individual patient, for a plurality of different patients;
a monitoring processor for monitoring data identifying orders initiated for treatment to be provided to an individual patient;
a data processor for generating data representing status of healthcare activity for a plurality of patients in response to said data identifying orders and said acuity data, for a plurality of different patients; and
an interface processor for providing said data representing status of healthcare activity to a healthcare worker via a display image identifying status of treatment related activity in different healthcare enterprise locations and for categories of operational characteristics comprising at least one of (a) capacity (b) acuity and (c) treatment delay.

10. A system according to claim 9, wherein
said monitoring processor monitors data identifying laboratory test results received for an individual patient, for a plurality of different patients and
said data processor generates data representing status of healthcare activity for a plurality of patients in response to said data identifying laboratory test results.

11. A system according to claim 9, wherein
said different categories of operational characteristics comprise at least one of, (a) delay in administering medication or intra-venous infusions, (b) patient discharge delays, (c) nurse call activity and (d) alerts generated.

12. A system according to claim 9, wherein
said monitoring data identifies orders initiated for treatment to be provided to an individual patient and data identifying laboratory and radiology test results and
said data processor generates data representing status of healthcare activity for a plurality of patients in response to said data identifying orders and laboratory and radiology test results.

13. A system according to claim 9, wherein
said interface processor generates data representing a composite single display image including data identifying status of treatment related activity in different healthcare enterprise locations and indicating available treatment related activity capacity.

14. A system according to claim 13, wherein
said composite single display image includes data indicating degrees of available treatment related activity capacity.

15. A system for monitoring activity in a healthcare enterprise, comprising:
an acquisition interface for acquiring acuity data representative of severity of medical condition of an individual patient, for a plurality of different patients;
a monitoring processor for monitoring data identifying laboratory test results received for an individual patient, for a plurality of different patients;
a data processor for generating data representing status of healthcare activity for a plurality of patients in response to said data identifying laboratory test results and said acuity data, for a plurality of different patients; and
an interface processor for providing said data representing status of healthcare activity to a healthcare worker via a display image identifying status of treatment related activity and indicating degrees of available treatment related activity capacity in different healthcare enterprise locations.

16. A system according to claim 15, wherein
said monitoring processor monitors data identifying orders initiated for treatment to be provided to an individual patient, for a plurality of different patients and
said data processor generates data representing status of healthcare activity for a plurality of patients in response to said data identifying orders, for a plurality of different patients.

17. A system according to claim 15, wherein
said interface processor generates data representing a composite single display image including data identifying status of treatment related activity in different healthcare enterprise locations and indicating degrees of available treatment related activity capacity.

18. A system according to claim 15, wherein
said display image identifies status of treatment related activity in different healthcare enterprise locations and for categories of operational characteristics comprising at least two of, (a) capacity, (b) acuity and (c) treatment delay.
